(19) United States
(12) Patent Application Publication
(10) Pub. No.: US 2024/0415428 A1
(43) Pub. Date: Dec. 19, 2024

(54) IMAGE PROCESSING APPARATUS, LIVING BODY OBSERVATION SYSTEM, AND IMAGE PROCESSING METHOD

(71) Applicant: OLYMPUS CORPORATION, Tokyo (JP)

(72) Inventors: Koki MORISHITA, Tokyo (JP); Hiromi TAKAHASHI, Tokyo (JP)

(73) Assignee: OLYMPUS CORPORATION, Tokyo (JP)

(21) Appl. No.: 18/817,446

(22) Filed: Aug. 28, 2024

Related U.S. Application Data
(63) Continuation of application No. PCT/JP2022/009331, filed on Mar. 4, 2022.

(51) Int. Cl. A61B 5/1455

(52) U.S. Cl. CPC A61B 5/14552

(57) ABSTRACT

An image processing apparatus includes a processor having hardware. The processor acquires a first image, a second image, and a third image of living tissue irradiated with first light, second light, and third light, respectively, calculates an index value that is correlated with a blood volume in the living tissue based on signal values of the first image and the second image, and calculates oxygen saturation of the living tissue based on the index value and a signal value of the third image. Each of the first light and the second light has a wavelength at which an absorption coefficient of oxygenated hemoglobin and an absorption coefficient of deoxygenated hemoglobin are the same, and the third light has a wavelength at which the absorption coefficients of the oxygenated hemoglobin and the absorption coefficient of the deoxygenated hemoglobin are different from each other.

### CROSS-REFERENCE TO RELATED APPLICATIONS

This is a continuation of International Application No. PCT/JP2022/009331, with an international filing date of Mar. 4, 2022, which is hereby incorporated by reference herein in its entirety.

### TECHNICAL FIELD

The present invention relates to an image processing apparatus, a living body observation system, and an image processing method.

### BACKGROUND ART

Conventionally, there is known a technique of measuring oxygen saturation of living tissue by using a difference in absorption coefficient between oxygenated hemoglobin (HbO2) and deoxygenated hemoglobin (Hb) (for example, see PTL 1). An endoscope system described in PTL 1 radiates, on living tissue, first measurement light at 470 nm+10 nm at which a difference between an absorption coefficient of HbO2 and an absorption coefficient of Hb is great, captures the first measurement light that is reflected by the living tissue, and calculates oxygen saturation based on a signal value of an acquired image. The oxygen saturation is a proportion of oxygenated hemoglobin in whole hemoglobin.

Intensity of the first measurement light that is reflected is dependent not only on the oxygen saturation but also on a blood volume in the living tissue. Accordingly, in PTL 1, the blood volume in the living tissue is measured using second measurement light in a red wavelength band from 590 nm to 700 nm that is affected by the blood volume, and calculates accurate oxygen saturation by using the measured blood volume.

### CITATION LIST

### Patent Literature

{PTL 1}
Publication of Japanese Patent No. 5766773

### SUMMARY OF THE INVENTION

An aspect of the present invention is an image processing apparatus including a processor including hardware, wherein the processor is configured to acquire a first image, a second image, and a third image of living tissue, wherein the first image is an image of the living tissue that is irradiated with first light having a first wavelength, the second image is an image of the living tissue that is irradiated with second light having a second wavelength different from the first wavelength, each of the first wavelength and the second wavelength is a wavelength at which an absorption coefficient of oxygenated hemoglobin and an absorption coefficient of deoxygenated hemoglobin are the same, and the third image is an image of the living tissue that is irradiated with third light having a third wavelength that is a wavelength at which the absorption coefficient of the oxygenated hemoglobin and the absorption coefficient of the deoxygenated hemoglobin are different from each other, wherein the processor is further configured to: calculate an index value that is correlated with a blood volume in the living tissue based on a first signal value of the first image and a second signal value of the second image; and calculate oxygen saturation of the living tissue based on the index value and a third signal value of the third image.

Another aspect of the present invention is a living body observation system including: a light source unit that outputs first light having a first wavelength, second light having a second wavelength different from the first wavelength, and third light having a third wavelength; an image capturing unit that captures a first image, a second image, and a third image of living tissue, wherein the first image is an image of the living tissue that is irradiated with the first light, the second image is an image of the living tissue that is irradiated with the second light, each of the first wavelength and the second wavelength is a wavelength at which an absorption coefficient of oxygenated hemoglobin and an absorption coefficient of deoxygenated hemoglobin are the same, and the third image is an image of the living tissue that is irradiated with the third light having a wavelength at which the absorption coefficient of the oxygenated hemoglobin and the absorption coefficient of the deoxygenated hemoglobin are different from each other; and wherein the living body observation system further comprises a processor that includes hardware and that is configured to process the first image, the second image, and the third image, wherein the processor is configured to: acquire the first image, the second image, and the third image; calculate an index value that is correlated with a blood volume in the living tissue based on a first signal value of the first image and a second signal value of the second image; and calculate oxygen saturation of the living tissue based on the index value and a third signal value of the third image.

Another aspect of the present invention is an image processing method including: acquiring a first image, a second image, and a third image of living tissue, wherein the first image is an image of the living tissue that is irradiated with first light having a first wavelength, the second image is an image of the living tissue that is irradiated with second light having a second wavelength different from the first wavelength, each of the first wavelength and the second wavelength is a wavelength at which an absorption coefficient of oxygenated hemoglobin and an absorption coefficient of deoxygenated hemoglobin are the same, and the third image is an image of the living tissue that is irradiated with third light having a third wavelength that is a wavelength at which the absorption coefficient of the oxygenated hemoglobin and the absorption coefficient of the deoxygenated hemoglobin are different from each other; wherein the image processing method further comprises: calculating an index value that is correlated with a blood volume in the living tissue based on a first signal value of the first image and a second signal value of the second image; and calculating oxygen saturation of the living tissue based on the index value and a third signal value of the third image.

### BRIEF DESCRIPTION OF THE DRAWINGS

FIG. 1 is an overall configuration diagram of a living body observation system according to an embodiment of the present invention.

FIG. 2 is a diagram showing a spectrum of light that is output by a light source device of the living body observation system in FIG. 1.

FIG. 3 is a graph showing a relationship between a wavelength and absorption coefficients of oxygenated hemoglobin (HbO2) and deoxygenated hemoglobin (Hb).

FIG. 4 is a graph showing a relationship between a wavelength and reflectance of living tissue at different blood volumes and at different oxygen saturation.

FIG. 5 is a graph showing a relationship between a first ratio and a relative blood volume.

FIG. 6 is a graph showing a relationship between a second ratio and oxygen saturation at different relative blood volumes.

FIG. 7 is a flowchart of an image processing method according to an embodiment of the present invention.

FIG. 8 is an overall configuration diagram of a modification of the living body observation system in FIG. 1.

FIG. 9 is a diagram showing a spectrum of light that is output by a light source device of the living body observation system in FIG. 8.

### DESCRIPTION OF EMBODIMENTS

Hereinafter, an image processing apparatus, a living body observation system, and an image processing method according to an embodiment of the present invention will be described with reference to the drawings.

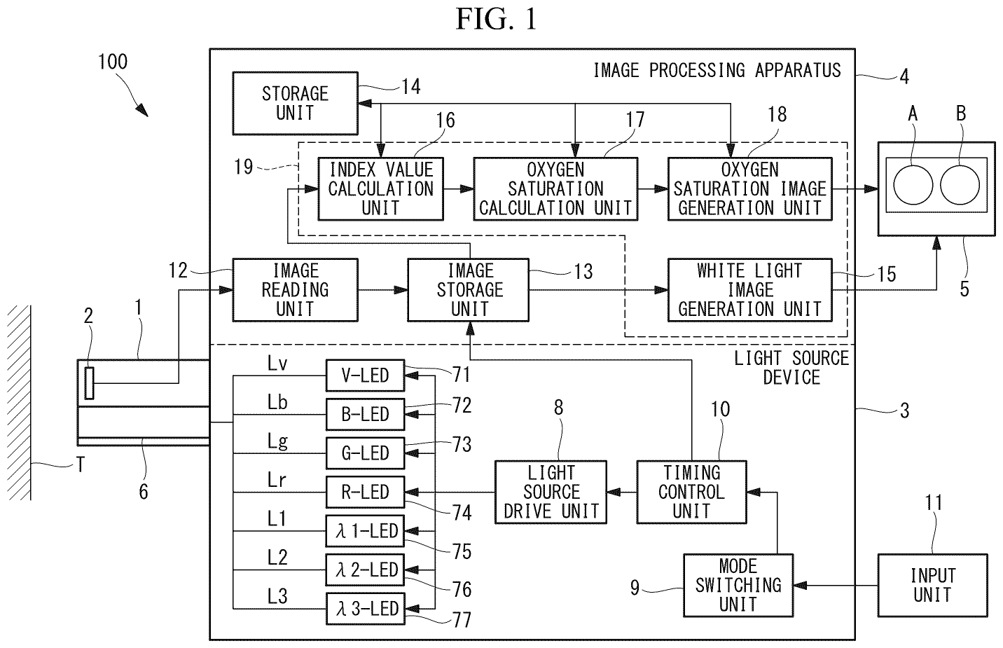

As shown in FIG. 1, a living body observation system 100 according to the present embodiment is an endoscope system that includes a long insertion section 1 to be inserted in a living body, and that generates a white light image A and an oxygen saturation image B of living tissue T.

The living body observation system 100 includes an image capturing unit 2 that captures the living tissue T, a light source device (a light source unit) 3 and an image processing apparatus (a processor) 4 that are connected to a proximal end of the insertion section 1, and a display 5 that is connected to the image processing apparatus 4 and that displays the white light image A and the oxygen saturation image B.

The image capturing unit 2 includes an image sensor such as a CCD image sensor or a CMOS image sensor, and is provided at a distal end portion of the insertion section 1. The image capturing unit 2 receives light that is reflected by the living tissue T, and captures an image of the living tissue T. The image capturing unit 2 may alternatively be provided on a proximal end side of the insertion section 1, and may capture an image that is transmitted from a distal end of the insertion section 1 to the image capturing unit 2 by a lens system or an image fiber.

The light source device 3 outputs purple light (V light) Lv, blue light (B light) Lb, green light (G light) Lg, and red light (R light) Lr for the white light image A. Furthermore, the light source device 3 outputs first light L1, second light L2, and third light L3 for the oxygen saturation image B. The beams of light Lv, Lb, Lg, Lr, L1, L2, and L3 output from the light source device 3 are guided to the distal end of the insertion section 1 by a light guide 6 provided in the insertion section 1, and is radiated on the living tissue T from the distal end of the insertion section 1.

More specifically, the light source device 3 includes seven LEDs 71, 72, 73, 74, 75, 76, and 77 that output the V light Lv, B light Lb, G light Lg, R light Lr, the first light L1, the second light L2, and the third light L3, respectively, a light source drive unit 8 that drives the LEDs 71, 72, 73, 74, 75, 76, and 77, a mode switching unit 9 that switches among image modes, and a timing control unit 10 that controls the light source drive unit 8 based on the image mode.

The light source drive unit 8, the mode switching unit 9, and the timing control unit 10 are implemented by a processor provided in the light source device 3, for example.

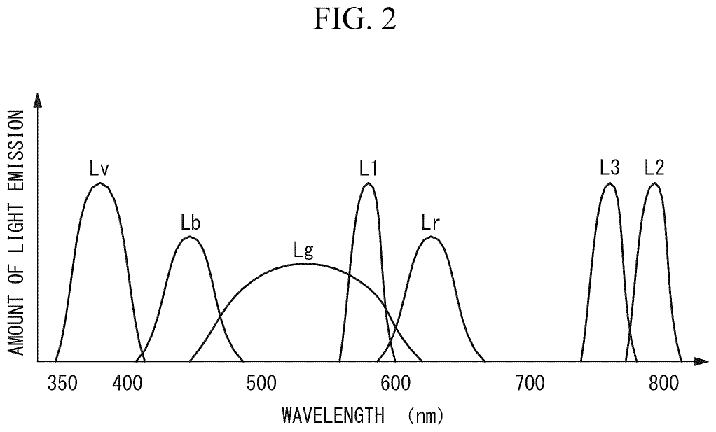

FIG. 2 shows spectra of the beams of light Lv, Lb, Lg, Lr, L1, L2, and L3 that are output by the LEDs 71, 72, 73, 74, 75, 76, and 77.

The first light L1 has a center wavelength at 584 nm, the second light L2 has a center wavelength at 796 nm, and the third light L3 has a center wavelength at 760 nm. The light L1, L2, and L3 may each be light having a single wavelength, or may be light having a wavelength width in a range of the center wavelength±5 nm, for example.

The mode switching unit 9 switches an image mode between a white light image mode in which the white light image A is generated, and an oxygen saturation image mode in which the oxygen saturation image B is generated, based on an input from a user. For example, the mode switching unit 9 is connected to an input unit 11 including input devices such as a mouse, a keyboard, and a touch panel. The user is able to switch between the white light image mode and the oxygen saturation image mode at any timing by using the input unit 11.

In the white light image mode, by controlling the light source drive unit 8, the timing control unit 10 causes the three LEDs 75, 76, and 77 to be turned off, and causes the four LEDs 71, 72, 73, and 74 to be sequentially turned on in synchronization with capturing by the image capturing unit 2. Accordingly, the V light Lv, the B light Lb, the G light Lg, and the R light Lr are sequentially radiated on the living tissue T in synchronization with timing of capturing by the image capturing unit 2, and a V image, a B image, a G image, and an R image are sequentially captured by the image capturing unit 2. The V image, the B image, the G image, and the R image are images of the living tissue T that is irradiated with the V light Lv, the B light Lb, the G light Lg, and the R light Lr, respectively.

In the white light image mode, the timing control unit 10 may alternatively cause the four LEDs 71, 72, 73, and 74 to be turned on at the same time. In this case, images of the living tissue T irradiated with the V light Lv, the B light Lb, the G light Lg, and the R light Lr are captured using a color image sensor.

In the oxygen saturation image mode, by controlling the light source drive unit 8, the timing control unit 10 causes the four LEDs 71, 72, 73, and 74 to be turned off, and causes the three LEDs 75, 76, and 77 to be sequentially turned on in synchronization with capturing by the image capturing unit 2. Accordingly, the first light L1, the second light L2, and the third light L3 are sequentially radiated on the living tissue T in synchronization with timing of capturing by the image capturing unit 2, and a first image, a second image, and a third image are sequentially acquired by the image capturing unit 2.

The first image, the second image, and the third image are images of the living tissue that is irradiated with the first light L1, the second light L2, and the third light L3, respectively. Each pixel in the first image has a first signal value P1 corresponding to reflection intensity of the first light L1 reflected at each position on the living tissue T. Each pixel in the second image has a second signal value P2 corresponding to reflection intensity of the second light L2 reflected at each position on the living tissue T. Each pixel in the third image has a third signal value P3 corresponding to reflection intensity of the third light L3 reflected at each position on the living tissue T.

The image processing apparatus 4 includes an image reading unit 12 that reads out an image from the image capturing unit 2, an image storage unit 13 that temporarily stores an image that is read out, a storage unit (a memory) 14 that stores programs and data necessary to generate the images A and B, a white light image generation unit 15 that generates the white light image A, an index value calculation unit 16 that calculates an index value V that is correlated with a blood volume in the living tissue T, an oxygen saturation calculation unit 17 that calculates oxygen saturation S of the living tissue T, and an oxygen saturation image generation unit 18 that generates the oxygen saturation image (a fourth image) B.

The image reading unit 12 successively reads out images from the image capturing unit 2, and stores the same in the image storage unit 13.

The image storage unit 13 transfers the image to the white light image generation unit 15 or the index value calculation unit 16, based on a signal from the timing control unit 10. That is, in the white light image mode, the image storage unit 13 transfers the V image, the B image, the G image, and the R image to the white light image generation unit 15. In the oxygen saturation image mode, the image storage unit 13 transfers the first image, the second image, and the third image to the index value calculation unit 16.

The storage unit 14 includes a working memory such as a RAM, and a non-volatile recording medium such as a ROM or an HDD, and an image processing program is stored in the recording medium.

The image processing apparatus 4 includes a processor 19 including hardware such as a central processing unit, and functions of the units 15, 16, 17, and 18 described later are implemented by the processor 19 performing processing according to the image processing program. A part of functions of the image processing apparatus 4 may be implemented by a dedicated circuit.

The white light image generation unit 15 generates the white light image A by combining the V image, the B image, the G image, and the R image.

The index value calculation unit 16 selects the signal values P1 and P2 of pixels at a same position from the first image and the second image, and calculates a first ratio P1/P2 between the first signal value P1 and the second signal value P2. The index value calculation unit 16 calculates the first ratio P1/P2 for pixels at all positions in the first image and the second image.

Next, the index value calculation unit 16 calculates the index value V from each ratio P1/P2. As described later, each index value V is a relative blood volume at each position in the living tissue T. The storage unit 14 stores reference data (first data) including a plurality of reference values and a plurality of relative blood volumes that are associated with the plurality of reference values, respectively. The index value calculation unit 16 compares each ratio P1/P2 with the plurality of reference values in the reference data, and calculates a relative blood volume that is associated with a reference value that is equal to or closest to the ratio P1/P2 as a relative blood volume V at a position of each pixel.

Characteristics of the first light L1 and the second light L2 will now be described.

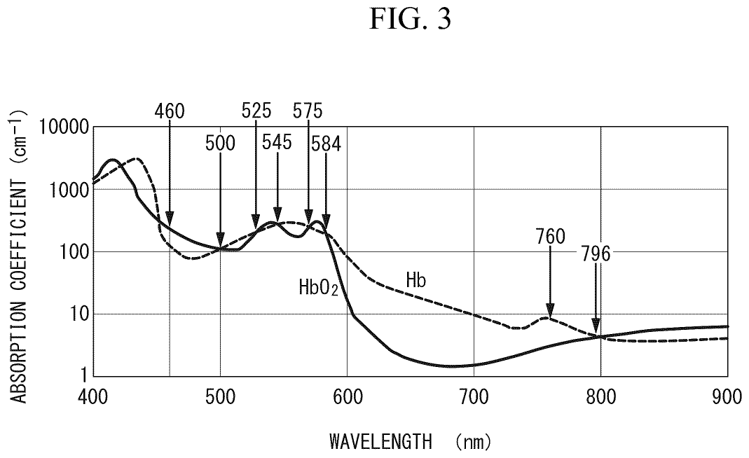

FIG. 3 shows a relationship between a wavelength and an absorption coefficient of each of oxygenated hemoglobin (HbO2) and deoxygenated hemoglobin (Hb).

Each of 584 nm and 796 nm is a wavelength at which the absorption coefficient of HbO2 and the absorption coefficient of Hb are the same. Accordingly, the reflection intensity of each of the first light L1 and the second light L2 reflected by the living tissue T is not dependent on the oxygen saturation of the living tissue T but is dependent on the blood volume in the living tissue T. Moreover, the reflection intensity of each of the light L1 and L2 changes according to a capturing distance from the distal end of the insertion section 1 to the living tissue T. By dividing the first signal value P1 by the second signal value P2, dependency on the capturing distance is eliminated, and the ratio P1/P2 that is dependent only on the blood volume is obtained. The ratio P1/P2 is correlated with the relative blood volume that is a relative volume of blood in the living tissue T. The relative blood volume is a proportion of an absolute blood volume of blood in the living tissue T based on absorption and scattering of light, or in other words, a proportion of amount of light that is absorbed and scattered by blood relative to an amount of light that is absorbed and scattered by the living tissue T.

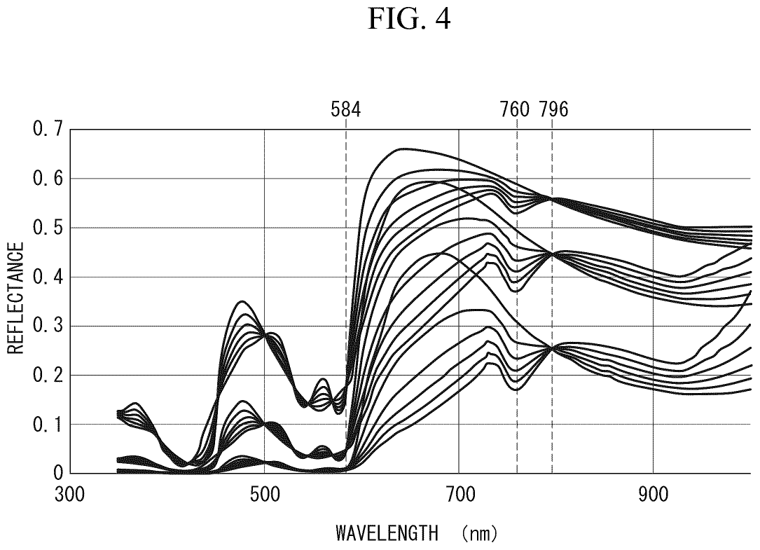

FIG. 4 shows a result of simulating a change in reflectance of light according to changes in the blood volume and the oxygen saturation. In the simulation, the blood volume is changed in five levels, and the oxygen saturation is changed in six levels in relation to each blood volume. In FIG. 4, simulation results for three blood volumes are shown as representatives.

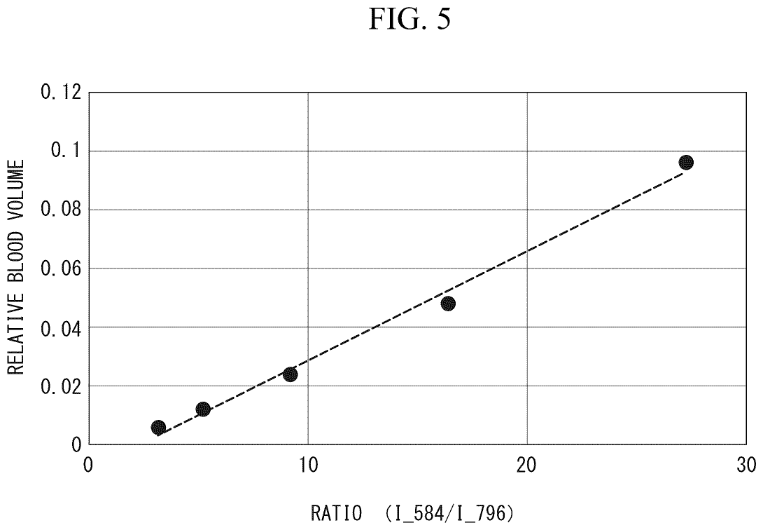

FIG. 5 shows a relationship between a ratio (I_584/1_796) and a relative blood volume obtained from the simulation results in FIG. 4. The ratio (I_584/1_796) is a ratio between reflectance I_584 at 584 nm and reflectance I_796 at 796 nm. As can be seen from FIG. 5, there is a substantially linear correlation between the relative blood volume and the ratio (I_584/1_796), and the relative blood volume is uniquely determined by the ratio (I_584/I_796). For example, a relationship between the ratio (I_584/1_796) and the relative blood volume is expressed by a linear or quadratic first approximate expression below. In the expressions below, X is the ratio (I_584/1_796), and Y is the relative blood volume.

Y=0.0037X−0.0083

Y=(5E−05)X\hat{ }2+0.0022X−0.0011

The signal values P1 and P2 correspond to the reflectance I_584 and I_796, respectively, and thus, the relative blood volume V can be calculated based on the ratio P1/P2. The aforementioned calculation is established at any two wavelengths at which the absorption coefficient of HbO2 and the absorption coefficient of Hb are the same.

The index value calculation unit 16 may calculate the relative blood volume V based on the ratio P1/P2 by using the linear or quadratic first approximate expression instead of the reference data. That is, the relative blood volume V is calculated by substituting the ratio P1/P2 in the X in the first approximate expression. In this case, the first approximate expression may be stored in the storage unit 14 in advance.

Generally, the reflection intensity is reduced as the blood volume in the living tissue T is increased, and is increased as scattering by the living tissue T is increased. The relative blood volume is a volume that takes into account the blood volume in the living tissue T and scattering by the living tissue T.

The oxygen saturation calculation unit 17 selects the signal values P3 and P2 of pixels at a same position from the third image and the second image, and calculates a second ratio P3/P2 between the third signal value P3 and the second signal value P2. The oxygen saturation calculation unit 17 calculates the second ratio P3/P2 for pixels at all positions in the third image and the second image.

Next, the oxygen saturation calculation unit 17 calculates an absolute value S of oxygen saturation at each position based on the ratio P3/P2 of the signal values of pixels at respective positions in respective images and the relative blood volume V.

Characteristics of the third light L3 will now be described.

The wavelength 760 nm is a wavelength at which the absorption coefficient of HbO2 and the absorption coefficient of Hb are different from each other. Accordingly, the reflection intensity of the third light L3 reflected by the living tissue T is dependent on the oxygen saturation and the blood volume of the living tissue T. Furthermore, as in the case of the first light L1 and the second light L2, the reflection intensity of the third light L3 changes according to the capturing distance. By dividing the third signal value P3 by the second signal value P2, dependency on the capturing distance and the blood volume is eliminated, and the ratio P3/P2 that is dependent only on the oxygen saturation is obtained.

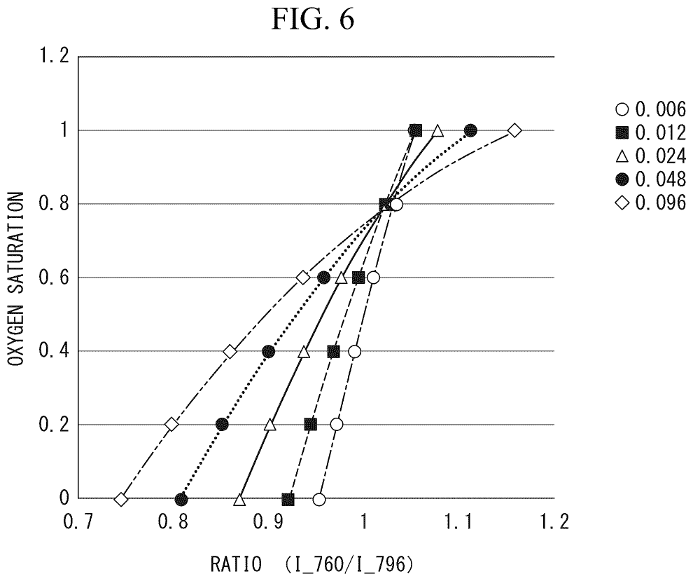

FIG. 6 shows a relationship between a ratio (I_760/1_796) and the oxygen saturation in relation to five relative blood volumes 0.006, 0.012, 0.024, 0.048, and 0.096 obtained from the simulation results in FIG. 4. The ratio (I_760/1_796) is a ratio between reflectance I_760 at 760 nm and the reflectance I_796 at 796 nm. As can be seen from FIG. 6, the oxygen saturation is monotonously increased in relation to the ratio (I_760/1_796), and a slope of increase is different in each relative blood volume. The signal values P3 and P2 correspond to the reflectance I_760 and I_796, respectively, and thus, the absolute value S of the oxygen saturation is uniquely determined based on the ratio P3/P2 and the relative blood volume V.

The oxygen saturation calculation unit 17 calculates the absolute value S of the oxygen saturation by a first method that uses a lookup table (second data) or a second method that uses a second approximate expression.

In the first method, the oxygen saturation calculation unit 17 calculates the absolute value S of the oxygen saturation based on a lookup table (LUT) that is prepared and stored in the storage unit 14 in advance.

The LUT includes a plurality of ratios P3/P2 and a plurality of oxygen saturations that are associated with the plurality of ratios P3/P2, respectively. An LUT for each relative blood volume is stored in the storage unit 14. The oxygen saturation calculation unit 17 selects a LUT for the relative blood volume V at each position of the pixel that is calculated, and calculates a value of the oxygen saturation that is associated with the calculated ratio P3/P2 in the selected LUT as the absolute value S of the oxygen saturation at each position.

The number of pieces of data about the relative blood volume V and the ratio P3/P2 that are stored in the LUT is finite. Accordingly, in the case where there is no data in the LUT that completely matches a combination of the relative blood volume V and the ratio P3/P2, data of values that are closest to the relative blood volume V and the ratio P3/P2 may be used in relation to the oxygen saturation image. For example, in the case where the relative blood volume V is 0.03 and the ratio P3/P2 is 0.95, oxygen saturation 0.4 corresponding to the combination of the relative blood volume V of 0.024 and the ratio P3/P2 of 0.95 may be selected.

In the second method, the oxygen saturation calculation unit 17 calculates the absolute value of the oxygen saturation by using the second approximate expression that is prepared and stored in the storage unit 14 in advance.

For example, in relation to each relative blood volume, the relationship between the oxygen saturation S and the ratio P3/P2 is expressed by a linear or quadratic second approximate expression as below. In the following expressions, X is the ratio P3/P2, and a, b, c, d, and e are constants that are set according to the relative blood volume.

Oxygen Saturation S=a*X+b

Oxygen Saturation S=c*X\hat{ }2+d*X+e

The second approximate expression indicating the relationship between the oxygen saturation S and the ratio P3/P2 in relation to each relative blood volume is stored in the storage unit 14. The oxygen saturation calculation unit 17 obtains the second approximate expression for the calculated relative blood volume V from the storage unit 14, and calculates the absolute value S of the oxygen saturation by substituting the ratio P3/P2 in the obtained second approximate expression.

There is a predetermined correlation between each constant a, b, c, d, or e and the relative blood volume. Accordingly, the oxygen saturation calculation unit 17 may determine the second approximate expression for the relative blood volume V by calculating the constant a, b or the constant c, d, e based on the relative blood volume V based on the predetermined correlation, and may calculate the absolute value S of the oxygen saturation by using the second approximate expression that is determined.

Additionally, either of the first image and the second image may be used for calculation of the oxygen saturation S. That is, the second ratio may be a ratio P3/P1 between the third signal value P3 and the first signal value P1.

Influence such as scattering that two beams of light receive from the living tissue T may differ due to a difference between wavelengths of the two beams of light. To suppress an error in the oxygen saturation S that is due to such a difference in influence, an image that uses light having a wavelength, between a first wavelength and a second wavelength, that is closer to a third wavelength is preferably used for calculation of the oxygen saturation S.

The oxygen saturation image generation unit 18 generates the oxygen saturation image B showing the oxygen saturation of the living tissue T, by assigning a signal value according to the absolute value S of the oxygen saturation to a pixel at each position. For example, a hue corresponding to the absolute value of each oxygen saturation is set in advance in such a way that the hue changes continuously from oxygen saturation 0% to 100%. Accordingly, a heat map where the absolute value S of the oxygen saturation at each position on the living tissue T is expressed by a color is generated as the oxygen saturation image B.

Next, an operation of the living body observation system 100 will be described.

The living body observation system 100 generates the white light image A or the oxygen saturation image B according to the image mode that is selected by the mode switching unit 9.

In the white light image mode, the V light, the B light, the G light, and the R light are sequentially radiated on the living tissue T due to the light source drive unit 8 being controlled by the timing control unit 10, and the V image, the B image, the G image, and the R image of the living tissue T are sequentially captured by the image capturing unit 2.

The V image, the B image, the G image, and the R image are sequentially read from the image capturing unit 2 into the image processing apparatus 4 by the image reading unit 12, and are temporarily stored in the image storage unit 13, and are then processed by the white light image generation unit 15. The white light image generation unit 15 generates the white light image by combining the V image, the B image, the G image, and the R image. The white light image is transmitted from the image processing apparatus 4 to the display 5, and is displayed by the display 5.

In the oxygen saturation image mode, the first light L1, the second light L2, and the third light L3 are sequentially radiated on the living tissue T due to the light source drive unit 8 being controlled by the timing control unit 10, and the first image, the second image, and the third image of the living tissue T are sequentially captured by the image capturing unit 2.

The first image, the second image, and the third image are sequentially read from the image capturing unit 2 into the image processing apparatus 4 by the image reading unit 12, and are temporarily stored in the image storage unit 13, and then, the oxygen saturation image B is generated from the first image, the second image, and the third image.

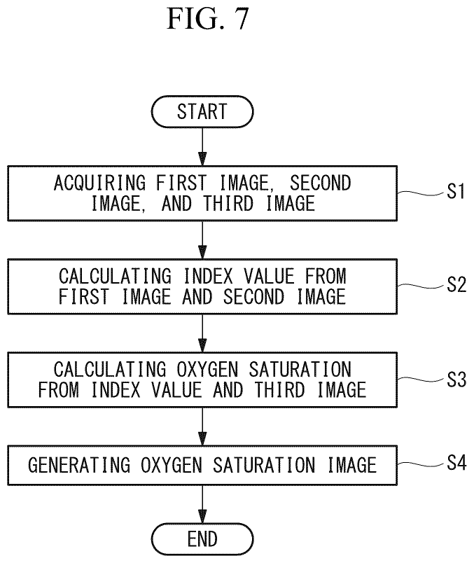

FIG. 7 shows an image processing method that is performed by the image processing apparatus 4 in the oxygen saturation image mode.

The image processing method includes step S1 of acquiring the first image, the second image, and the third image, step S2 of calculating the index value V that is correlated with the blood volume in the living tissue T from the first image and the second image, step S3 of calculating the oxygen saturation S of the living tissue T from the index value V and the third image, and step S4 of generating the oxygen saturation image B indicating the oxygen saturation S of the living tissue T.

In step S1, the processor 19 acquires the first image, the second image, and the third image captured by the image capturing unit 2, via the image storage unit 13 and the image reading unit 12.

Next, in step S2, the ratio P1/P2 between signal values at each position of the pixel is calculated by the index value calculation unit 16 from the first image and the second image, and then, the relative blood volume V at each position of the pixel is calculated as the index value based on the ratio P1/P2 by using the reference data or the first approximate expression.

Next, in step S3, the ratio P3/P2 between signal values at each position of the pixel is calculated by the oxygen saturation calculation unit 17 from the second image and the third image, and then, the absolute value S of the oxygen saturation at each position of the pixel is calculated based on the ratio P3/P2 by using the LUT for the relative blood value V calculated in step S2 or the second approximate expression.

Next, in step S4, the oxygen saturation image B where the signal value corresponding to the absolute value S of the oxygen saturation is assigned to the pixel at each position is generated by the oxygen saturation image generation unit 18.

The oxygen saturation image B is transmitted from the image processing apparatus 4 to the display 5, and is displayed by the display 5. The user may intuitively check the absolute value of the oxygen saturation at each position on the living tissue T based on the signal value (such as a hue) at each position in the oxygen saturation image B.

Clinically, it is desired that the absolute value of the oxygen saturation is measured such that a state of the living tissue T can be accurately grasped. For example, it is known that cancer has lower oxygen saturation compared to surrounding parts. Accordingly, in an endoscopic examination of a digestive organ, visualization of the oxygen saturation is expected to enable visualization of a cancer region. Moreover, in a surgery, visualization of the oxygen saturation is expected to enable visualization of a tissue region that is dominated by a blood vessel that is sealed by forceps or the like.

However, the reflection intensity of light that is reflected by the living tissue T is dependent also on the blood volume and the capturing distance, and thus, it is difficult to calculate the oxygen saturation directly from information about the reflection intensity of light.

According to the present embodiment, the first image and the second image are captured by using the light L1 and L2 at the wavelengths 584 nm and 796 nm that are not affected by the oxygen saturation, and the third image is acquired by using the light L3 at the wavelength 760 nm that is affected by the oxygen saturation.

The first signal value P1 of the first image and the second signal value P2 of the second image are not dependent on the oxygen saturation but are dependent on the blood volume. Accordingly, the index value V that is correlated with the blood volume in the living tissue T can be obtained from the signal values P1, P2.

Particularly, by dividing the first signal value P1 by the second signal value P2, the dependency on the capturing distance is eliminated, and the first ratio P1/P2 that is accurately correlated with the blood volume can be obtained. An accurate relative blood volume of the living tissue T can be calculated as the index value V by such a first ratio P1/P2.

Furthermore, the third signal value P3 of the third image is dependent on both the oxygen saturation and the blood volume. Accordingly, influence of the blood volume on calculation of the oxygen saturation may be corrected by using the accurate index value V of the blood volume, and accurate oxygen saturation S can be calculated from the third signal value P3.

Particularly, by dividing the third signal value P3 by the second signal value P2, dependency on both the blood volume and the capturing distance is eliminated, and the second ratio P3/P2 that is accurately correlated with the oxygen saturation can be obtained. A more accurate absolute value S of the oxygen saturation can be calculated based on such a second ratio P3/P2 and the index value V.

Furthermore, by simply adding the three light sources 75, 76, and 77 to a general light source device for endoscope, the first image, the second image, and the third image necessary for calculation of the oxygen saturation S can be acquired. Accordingly, the image processing apparatus 4 and the image processing method of the present embodiment may be suitably applied to an endoscope system.

In the present embodiment, the first wavelength of the first light L1 is 584 nm, and the second wavelength of the second light L2 is 796 nm, but the combination of the first wavelength and the second wavelength is not limited to such an example, and may be a combination of any two wavelengths at which the absorption coefficient of HbO2 and the absorption coefficient of Hb are the same.

For example, the first wavelength and the second wavelength may be selected from around 460 nm, around 500 nm, around 525 nm, around 545 nm, around 575 nm, around 584 nm, and around 820 nm.

The first wavelength and the second wavelength are preferably 500 nm or more.

A surface of the living tissue T may be covered with fat tissue including β-carotene. β-carotene performs absorption in a wavelength band less than 500 nm, but hardly performs absorption in a wavelength band equal to or greater than 500 nm. Accordingly, by using the first wavelength and the second wavelength at or greater than 500 nm, the signal values P1, P2 that are not dependent on presence/absence of fat tissue and a thickness of the fat tissue can be obtained, and more accurate relative blood volume V and more accurate oxygen saturation S can be calculated.

In the present embodiment, the third wavelength is 760 nm, but the third wavelength is not limited to such an example, and may be any other wavelength at which the absorption coefficient of HbO2 and the absorption coefficient of Hb are different from each other.

Preferably, the third wavelength is a wavelength at which a difference between the absorption coefficient of HbO2 and the absorption coefficient of Hb is great. Furthermore, as with the first wavelength and the second wavelength, the third wavelength is preferably equal to or greater than 500 nm at which influence of fat is not easily received.

For example, the third wavelength may be around 630 nm. This allows the LED 71 for the R light Lr to be used as a light source for the third light L3, and the number of light sources mounted on the light source device 3 can be reduced.

In the embodiment described above, the light source device 3 includes dedicated light sources 75, 76, and 77 for the first light L1, the second light L2, and the third light L3, but instead, a light source that is mounted as standard in an endoscope may be used.

According to such a configuration, the number of light sources mounted on the light source device 3 can be reduced.

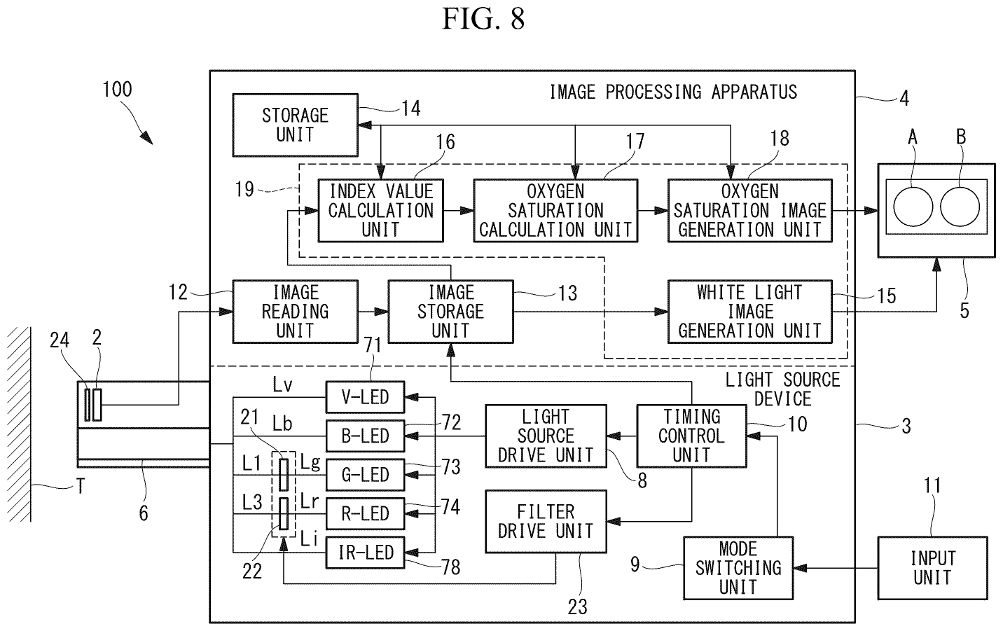

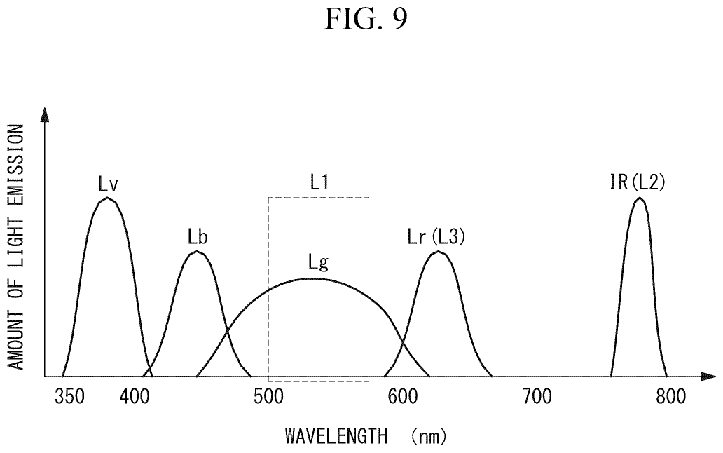

For example, as shown in FIGS. 8 and 9, the G light Lg output from the G-LED 73 may be used as the first light L1, and the R light Lr output from the R-LED 74 may be used as the third light L3.

In this case, a bandpass filter 21, 22 that sets a wavelength width of the light L1, L3 is removably disposed on an optical path between each light source 73, 74 and the light guide 6.

The first filter 21 has a center wavelength at 584 nm, and generates the first light L1 from the G light Lg. The second filter 22 has a center wavelength at 630 nm, and generates the third light L3 from the R light Lr. In the white light image mode, the timing control unit 10 removes the filters 21, 22 from the optical paths by controlling a filter drive unit 23, and in the oxygen saturation image mode, the timing control unit 10 disposes the filters 21, 22 on the optical paths by controlling the filter drive unit 23.

As shown in FIG. 9, the wavelength width of the first light L1 (a rectangular range surrounded by a dashed line) is limited by the second filter 22 in such a way that an amount of absorption of the first light L1 by HbO2 and an amount of absorption by Hb are made equal to each other (or in other words, in such a way that an integral value of the absorption coefficient of HbO2 and an integral value of the absorption coefficient of Hb are made equal to each other in the wavelength width of the first light L1). The wavelength width may be set by further taking into account spectral transmittance characteristics of a red color filter of the image capturing unit 2.

In the case where the light source device 3 includes a near-infrared light source 78 for excitation of indocyanine green (ICG), near-infrared light Li that is output from the near-infrared light source 78 may be used as the second light L2.

ICG is a fluorescent substance that is injected into a blood vessel for evaluation of a blood flow. A cut filter 24 that cuts off the near-infrared light Li is removably disposed in front of the image capturing unit 2. The cut filter 24 is disposed in front of the image capturing unit 2 in a fluorescence image mode in which a fluorescence image of ICG is generated, and is removed in the oxygen saturation image mode.

According to such a configuration, a state of the blood flow in the living tissue T may be evaluated by using both fluorescence of ICG and the oxygen saturation S. For example, in the case where it is difficult to determine the state of the blood flow in the living tissue T from the fluorescence image of ICG, the state of the blood flow may be determined based on the oxygen saturation S by switching from the fluorescence image mode to the oxygen saturation image mode.

In the embodiment described above, a signal value corresponding to the oxygen saturation S is assigned to every pixel in the oxygen saturation image B, but the signal value may instead be assigned to a partial region in the oxygen saturation image B so that the oxygen saturation image B showing only the partial region is generated.

For example, in the case where an observation target is a specific organ or tissue, the observation target may be extracted based on at least one of the signal values P1, P2, and P3 or based on color of the white light image, and the index value V and the oxygen saturation S may be calculated and a signal value may be assigned only in relation to a position in the extracted region.

In the embodiment described above, the light source device 3 includes the LEDs 71, 72, 73, 74, 75, 76, 77, and 78 as light sources, but instead, other types of light sources may be provided. For example, the light source may be a laser light source such as a laser diode (LD), or a lamp light source such as a xenon lamp that is used in combination with a bandpass filter.

In the embodiment described above, the living body observation system 100 switches between the white light image mode and the oxygen saturation image based on an input from the user, but instead, switching between the white light image mode and the oxygen saturation image may be automatically performed at a predetermined timing. For example, the living body observation system 100 may alternately generate the white light image and the oxygen saturation image by alternately switching between the white light image mode and the oxygen saturation image mode.

In the embodiment described above, the living body observation system 100 is an endoscope system, but the living body observation system may be any type of system that acquires an optical image of living tissue. For example, the living body observation system may be a microscope system including an optical microscope that is used to observe inside of a living body.

The present disclosure is advantageous in which an influence of a blood volume in calculation of oxygen saturation can be corrected, and accurate oxygen saturation can be calculated.

### REFERENCE SIGNS LIST

2 image capturing unit
3 light source device (light source unit)
4 image processing apparatus (processor)
14 storage unit (memory)
19 processor
100 living body observation system
A white light image
B oxygen saturation image (fourth image)
L1 first light
L2 second light
L3 third light
T living tissue

1. An image processing apparatus comprising a processor including hardware, wherein
the processor is configured to acquire a first image, a second image, and a third image of living tissue, wherein
the first image is an image of the living tissue that is irradiated with first light having a first wavelength,
the second image is an image of the living tissue that is irradiated with second light having a second wavelength different from the first wavelength,
each of the first wavelength and the second wavelength is a wavelength at which an absorption coefficient of oxygenated hemoglobin and an absorption coefficient of deoxygenated hemoglobin are the same, and
the third image is an image of the living tissue that is irradiated with third light having a third wavelength that is a wavelength at which the absorption coefficient of the oxygenated hemoglobin and the absorption coefficient of the deoxygenated hemoglobin are different from each other,
wherein the processor is further configured to:
calculate an index value that is correlated with a blood volume in the living tissue based on a first signal value of the first image and a second signal value of the second image; and
calculate oxygen saturation of the living tissue based on the index value and a third signal value of the third image.

2. The image processing apparatus according to claim 1, wherein the processor is configured to:
calculate a first ratio between the first signal value and the second signal value, and
calculate, as the index value, a relative blood volume in the living tissue based on the calculated first ratio.

3. The image processing apparatus according to claim 2, further comprising a memory, wherein first data including a plurality of reference values and a plurality of relative blood volumes that are associated with the plurality of reference values, respectively, is stored in the memory, wherein the processor calculates the relative blood volume based on the first ratio and using the first data.

4. The image processing apparatus according to claim 2, wherein the processor calculates the relative blood volume based on the first ratio that is calculated by using a first approximate expression indicating a relationship between the first ratio and the relative blood volume.

5. The image processing apparatus according to claim 2, further comprising a memory, wherein second data including a plurality of second ratios and a plurality of oxygen saturations that are associated with the plurality of second ratios, respectively, is stored in the memory,
wherein the second ratio is a ratio between the third signal value and one of the first signal value and the second signal value, and
wherein the processor is configured to:
calculate the second ratio; and
calculate the oxygen saturation based on the calculated second ratio and using the second data.

6. The image processing apparatus according to claim 2, wherein the processor is configured to:
calculate a second ratio between the third signal value and one of the first signal value and the second signal value; and
calculate the oxygen saturation based on the calculated second ratio and using a second approximate expression indicating a relationship between the oxygen saturation and the second ratio for the calculated relative blood volume.

7. The image processing apparatus according to claim 1, wherein the processor generates a fourth image showing the oxygen saturation of the living tissue.

8. The image processing apparatus according to claim 1, wherein the first wavelength and the second wavelength are each equal to or greater than 500 nm.

9. The image processing apparatus according to claim 1, wherein the third wavelength is equal to or greater than 500 nm.

10. The image processing apparatus according to claim 8, wherein
the first wavelength is 584 nm, and
the second wavelength is 796 nm.

11. The image processing apparatus according to claim 9, wherein the third wavelength is 760 nm.

12. A living body observation system comprising:
a light source unit that outputs first light having a first wavelength, second light having a second wavelength different from the first wavelength, and third light having a third wavelength;
an image capturing unit that captures a first image, a second image, and a third image of living tissue, wherein
the first image is an image of the living tissue that is irradiated with the first light,
the second image is an image of the living tissue that is irradiated with the second light,
each of the first wavelength and the second wavelength is a wavelength at which an absorption coefficient of oxygenated hemoglobin and an absorption coefficient of deoxygenated hemoglobin are the same, and
the third image is an image of the living tissue that is irradiated with the third light having a wavelength at which the absorption coefficient of the oxygenated hemoglobin and the absorption coefficient of the deoxygenated hemoglobin are different from each other; and
wherein the living body observation system further comprises a processor that includes hardware and that is configured to process the first image, the second image, and the third image, wherein
the processor is configured to:
acquire the first image, the second image, and the third image;
calculate an index value that is correlated with a blood volume in the living tissue based on a first signal value of the first image and a second signal value of the second image; and
calculate oxygen saturation of the living tissue based on the index value and a third signal value of the third image.

13. The living body observation system according to claim 12, wherein the first wavelength and the second wavelength are each equal to or greater than 500 nm.

14. The living body observation system according to claim 12, wherein the third wavelength is equal to or greater than 500 nm.

15. The living body observation system according to claim 13, wherein
the first wavelength is 584 nm, and
the second wavelength is 796 nm.

16. The living body observation system according to claim 14, wherein the third wavelength is 760 nm.

17. An image processing method comprising
acquiring a first image, a second image, and a third image of living tissue, wherein
the first image is an image of the living tissue that is irradiated with first light having a first wavelength,
the second image is an image of the living tissue that is irradiated with second light having a second wavelength different from the first wavelength,
each of the first wavelength and the second wavelength is a wavelength at which an absorption coefficient of oxygenated hemoglobin and an absorption coefficient of deoxygenated hemoglobin are the same, and
the third image is an image of the living tissue that is irradiated with third light having a third wavelength that is a wavelength at which the absorption coefficient of the oxygenated hemoglobin and the absorption coefficient of the deoxygenated hemoglobin are different from each other;
wherein the image processing method further comprises:
calculating an index value that is correlated with a blood volume in the living tissue based on a first signal value of the first image and a second signal value of the second image; and
calculating oxygen saturation of the living tissue based on the index value and a third signal value of the third image.

18. The image processing method according to claim 17, wherein the first wavelength and the second wavelength are each equal to or greater than 500 nm.

19. The image processing method according to claim 17, wherein the third wavelength is equal to or greater than 500 nm.

20. The image processing method according to claim 18, wherein
the first wavelength is 584 nm, and
the second wavelength is 796 nm.
